(19) United States
(12) Patent Application Publication
(10) Pub. No.: US 2026/0109936 A1
(43) Pub. Date: Apr. 23, 2026

(54) A METHOD FOR PREPARING AND SELECTING A POLYMERIC FLOCCULANT FOR USE IN HARVESTING MICROALGAE

(71) Applicant: UNIVERSITY OF TECHNOLOGY SYDNEY, Ultimo NSW (AU)

(72) Inventors: Long D NGHIEM, Ultimo NSW (AU); Peter RALPH, Ultimo NSW (AU); Luong N NGUYEN, Ultimo NSW (AU); Qiang FU, Ultimo NSW (AU)

(21) Appl. No.: 19/117,358

(22) PCT Filed: Sep. 28, 2023
(86) PCT No.: PCT/AU2023/050941

§ 371 Date: Mar. 31, 2025

(30) Foreign Application Priority Data
Sep. 30, 2022 (AU) 2022902832

(51) Int. Cl. C12N 1/12; C08F 20/34; C12M 1/00

(52) U.S. Cl. CPC C12N 1/12; C08F 20/34; C12M 47/02

(57) ABSTRACT

The present invention relates to a method for harvesting microalgae, the method comprising (i) mixing one or more monomers, a photoinitiator, water and a deoxygenation system to form a mixture; (ii) exposing the mixture to UV light so as to form an aqueous solution of one or more polymers; (iii) introducing the aqueous solution of one or more polymers to a suspension comprising microalgae in an amount sufficient to produce flocculated microalgae; and (iv) harvesting the flocculated microalgae. The present invention also relates to a method for preparing and selecting a polymeric flocculant for use in harvesting microalgae and a related apparatus.

### CROSS-REFERENCE TO RELATED APPLICATIONS

The present application claim priority to Australian Provisional Application No. 2022902832 filed 30 Sep. 2022, the entire contents of which is incorporated herein by cross-reference.

### FIELD OF THE DISCLOSURE

The present disclosure broadly relates to a method for preparing and selecting a polymeric flocculant for use in flocculating suspended particles, particularly harvesting microalgae, and to an apparatus for performing the method.

### BACKGROUND OF THE DISCLOSURE

Any discussion of the prior art throughout this specification should in no way be considered as an admission that such prior art is widely known or forms part of the common general knowledge in the field.

Microalgae continues to receive considerable interest as a result of its extensive potential applications in the renewable energy, biopharmaceutical and nutraceutical fields.

Microalgae offer renewable and sustainable sources of biofuels, medicines and food ingredients. Microalgae-derived biomass is capable of supplying a wide range of biofuels including biodiesel, bioethanol, biohydrogen, biomethane and bioelectricity. Microalgae are an excellent platform for capturing carbon dioxide and converting it to useful products such as carbohydrates, lipids and other bioactive metabolites. Once cultivated, microalgal cells must be separated from the cultivation solution for further processing. Microalgal cells are small (˜50 μm) and negatively charged, and as a result can remain in solution as individual cells without aggregation.

Conventional methods of solid-liquid separation are unable to be applied directly to microalgal solutions due to their stabilised suspension properties. Microalgae are very fine and remain separated from each other in order to optimise their photosynthetic activity for growth. Artificial cultivation of microalgae for biomass or management of algal blooms needs to effectively remove the algae from culture. Flocculation is a process that involves adding a flocculant to destabilise the microalgal suspension into larger flocs thereby permitting solid-liquid separation.

With over 200,000 recognised species, microalgae are highly diverse in both their habitats (i.e. freshwater to seawater) as well as their physiochemical properties (i.e. cell size, shape, metabolites). This diversity is such that a single flocculant is not effective across different microalgal species and environments. As a result, different flocculants are required in order to effectively and efficiently harvest different microalgae depending on their type and environment.

Furthermore, polymeric flocculants cannot be added directly to a suspension to be flocculated, e.g. a suspension of microalgae, as a solid. The flocculant must be provided in the form of an aqueous solution of a suitable concentration. Conventionally, this flocculant solution is prepared in a batch process. However, this has significant disadvantages. In a batch process, a predetermined amount of a solid polymer is dissolved in water, and then diluted to a suitable concentration for addition to the suspension to be flocculated. This is a multi-step, space-intensive process, which involves working with hazardous materials (powders). It is not possible to carry out such a batch process on location. Furthermore, once produced, the polymer solution must be used within a certain time before it becomes ineffective for flocculation due to hydrolysis (degradation of the polymer).

### SUMMARY OF THE DISCLOSURE

In a first aspect of the disclosure there is provided a method for harvesting microalgae, the method comprising:
(i) mixing one or more monomers, a photoinitiator, water and a deoxygenation system to form a mixture;
(ii) exposing the mixture to UV light so as to form an aqueous solution of one or more polymers;
(iii) introducing the aqueous solution of one or more polymers to a suspension comprising microalgae in an amount sufficient to produce flocculated microalgae; and
(iv) harvesting the flocculated microalgae.

The below features may be used in combination with the first aspect of the disclosure, alone or in any combination.

Steps (i) and (ii) may take place in a flow reactor. The method may be a continuous method.

The one or more monomers may be compounds comprising an alkene and a cationic moiety. The one or more monomers may be selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC) and [3-(methacryloylamino)propyl]trimethylammonium chloride (MAmPTAC). The one or more monomers may be [2-(acryloyloxy)ethyl]trimethylammoniumchloride (AETAC).

Step (i) may further comprise mixing one or more co-monomers with the one or more monomers, the photoinitiator, the water and the deoxygenation system to form a mixture. The one or more co-monomers may be selected from the group consisting of methyl acrylate, methyl methacrylate, acrylamide, styrene, vinyl chloride, acrylonitrile and 2-(dimethylamino)ethyl acrylate (DMA).

The photoinitiator may be selected from the group consisting of azobisisobutyronitrile (AIBN), benzoyl peroxide (BPO), 2,2-dimethoxy-2-phenylacetophenone (DMPA), camphorquinone (CQ), 2,2′-azobis(2-methylpropionamidine)dihydrochloride (V-50) and 4,4′-azobis(4-cyanovaleric acid) (V-501).

The deoxygenation system may be an enzymatic deoxygenation system. The enzymatic deoxygenation system may comprise an enzyme and an enzyme substrate. In this case, the enzyme may be glucose oxidase and the enzyme substrate may be glucose.

Harvesting the flocculated microalgae may comprise filtering the suspension comprising microalgae to separate the flocculated microalgae.

The method may take place in situ.

In a second aspect of the disclosure there is provided a method for preparing and selecting a polymeric flocculant for use in harvesting microalgae, the method comprising:
(i) preparing a plurality of different polymers;
(ii) introducing an amount of each polymer into a separate suspension comprising microalgae;
(iii) repeating step (ii) at least once by varying the amount of each polymer so as to provide a series of separate suspensions comprising different polymers in different amounts;
(iv) measuring optical density of each suspension in the series;
(v) calculating a flocculation efficiency for each suspension in the series;
(vi) generating a flocculation efficiency dose-response curve for each polymer at each amount; and
(vii) selecting a polymeric flocculant.

The below features may be used in combination with the second aspect of the disclosure, alone or in any combination.

In step (vii) the polymeric flocculant may be selected based on the dose-response curves,

Steps (i) to (iii) may take place in situ.

Step (ii) may be repeated at least 2, 3, 4, 5, 6, 7, 8, 9 or 10 times.

The different polymers may differ in molecular weight, composition and/or charge density.

In step (i), each of the plurality of different polymers may be prepared by:
a. mixing one or more monomers, a photoinitiator, water and a deoxygenation system to form a mixture; and
b. exposing the mixture to UV light so as to form an aqueous solution of the polymer;
   wherein in step (ii), introducing an amount of each polymer into a separate suspension comprising microalgae comprises introducing an amount of the aqueous solution of the polymer.

When each of the plurality of different polymers are prepared as above, steps a. and b. may take place in a flow reactor.

When each of the plurality of different polymers are prepared as above, the one or more monomers may be compounds comprising an alkene and a cationic moiety. The one or more monomers may be selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC) and [3-(methacryloylamino)propyl]trimethylammonium chloride (MAmPTAC). The one or more monomers may be [2-(acryloyloxy)ethyl]trimethylammoniumchloride (AETAC).

When each of the plurality of different polymers are prepared as above, step a. may further comprise mixing one or more co-monomers with the one or more monomers, the photoinitiator, the water and the deoxygenation system to form a mixture. The one or more co-monomers may be selected from the group consisting of: methyl acrylate, methyl methacrylate, acrylamide, styrene, vinyl chloride, acrylonitrile, and 2-(dimethylamino)ethyl acrylate (DMA).

When each of the plurality of different polymers are prepared as above, the photoinitiator may be selected from the group consisting of azobisisobutyronitrile (AIBN), benzoyl peroxide (BPO), 2,2-dimethoxy-2-phenylacetophenone (DMPA), camphorquinone (CQ), 2,2′-azobis(2-methylpropionamidine)dihydrochloride (V-50) and 4,4′-azobis(4-cyanovaleric acid) (V-501).

When each of the plurality of different polymers are prepared as above, the deoxygenation system may be an enzymatic deoxygenation system. The enzymatic deoxygenation system may comprise an enzyme and an enzyme substrate. In this case, the enzyme may be glucose oxidase and the enzyme substrate may be glucose.

Step (v) may comprise calculating the flocculation efficiency according to the following equation:

$$Flocculation efficiency ( \% ) = ( \frac{{OD}_{i} - {OD}_{f}}{{OD}_{i}} ) \times 100$$

wherein ODᵢ is the optical density of the suspension prior to introduction of the polymer in step (ii), and OD_{f} is the optical density of the suspension after introduction of the polymer in step (ii).

In a third aspect of the disclosure there is provided an apparatus for flocculating suspended particles using one or more polymers, the apparatus comprising:
a plurality of vessels for containing one or more monomers, a photoinitiator, water and a deoxygenation system;
a mixing vessel for mixing the one or more monomers, the photoinitiator, the water and the deoxygenation system to form a mixture;
a polymerisation vessel for producing a polymer;
a UV light source for irradiating the polymerisation vessel; and
   wherein the plurality of vessels are in fluid communication with the mixing vessel, and the mixing vessel is in fluid communication with the polymerisation vessel.

The below features may be used in combination with the third aspect of the disclosure, alone or in any combination.

The apparatus may further comprise a first pump for transferring the one or more monomers, the photoinitiator, the water and the deoxygenation system from the plurality of vessels to the mixing vessel. The first pump for transferring the one or more monomers, the photoinitiator, the water and the deoxygenation system from the plurality of vessels to the mixing vessel may be a positive displacement pump. The first pump for transferring the one or more monomers, the photoinitiator, the water and the deoxygenation system from the plurality of vessels to the mixing vessel may be a peristaltic pump.

The apparatus may further comprise a second pump for transferring the aqueous solution of the polymer from the polymerisation vessel. The second pump may be for transferring the aqueous solution of the polymer from the polymerisation vessel to a body of water. Alternatively, the apparatus may further comprise a flocculation vessel for containing a suspension of particles, wherein the polymerisation vessel is in fluid communication with the flocculation vessel, and the second pump may be for transferring the aqueous solution of the polymer from the polymerisation vessel to the flocculation vessel. The second pump may be a dosing pump.

The polymerisation vessel may be in the form of a tube which is permeable to UV radiation.

The polymerisation vessel and the UV light source may be located within a container which excludes visible light.

The suspended particles may be microalgae.

In a fourth aspect of the disclosure there is provided a use of the apparatus of the third aspect of the disclosure for flocculating suspended particles.

In a fifth aspect of the disclosure there is provided the apparatus of the third aspect of the disclosure when used for flocculating suspended particles.

The below features may be used in combination with the fourth and/or fifth aspects of the disclosure, alone or in any combination.

The suspended particles may be selected from the group consisting of mud, silt, clay, microalgae, mine tailings, and particles found in wastewater.

The suspended particles may be microalgae.

### Definitions

The following are some definitions that may be helpful in understanding the description of the present disclosure. These are intended as general definitions and should in no way limit the scope of the present disclosure to those terms alone, but are put forth for a better understanding of the following description.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The terms “a” and “an” are used herein to refer to one or to more than one (i.e. to at least one) of the grammatical object of the article. By way of example, “an element” means one element or more than one element.

In the context of this specification the term “about” is understood to refer to ±10% of the recited value.

Any numerical range recited herein is intended to include all sub-ranges of the same numerical precision subsumed within the recited range. For example, a range of 1.0 to 5.0 is intended to include all sub-ranges between (and including) the recited minimum value of 1.0 and the recited maximum value of 5.0, that is, having a minimum value equal to or greater than 1.0 and a maximum value equal to or less than 5.0, such as 2.1 to 4.5. Any maximum numerical limitation recited herein is intended to include all lower numerical limitations subsumed therein and any minimum numerical limitation recited herein is intended to include all higher numerical limitations subsumed therein.

### BRIEF DESCRIPTION OF THE DRAWINGS

FIG. 1: An apparatus in accordance with one embodiment of the disclosure.

FIG. 2: Flocculation efficiency of three polymers based on AETAC with Chlorella vulgaris.

FIG. 3: Flocculation efficiency of three polymers based on AETAC with Scenedesmus sp.

FIG. 4: Flocculation efficiency of three polymers based on AETAC with Rhodomonas salina.

FIG. 5: Flocculation efficiency of three polymers based on AETAC with Porphyridium purpureum.

FIG. 6: Biomass floc formation as observed visually after adding polymer to culture of Chlorella vulgaris, Scenedesmus sp, Rhodomonas salina, and Porphyridium purpureum.

FIG. 7: Schematic of an embodiment of the second aspect of the disclosure.

### DETAILED DESCRIPTION

Optimisation of flocculant types and doses have previously been determined as key requirements for lowering production costs associated with microalgal harvesting. One aspect of the disclosure involves the convenient preparation of a range of different polymer flocculants followed by addition of different amounts of each polymer flocculant into a microalgal suspension, with a view to determining the optimal polymer flocculant and amount for use in harvesting applications. The method permits the preparation and identification of “fit-for-purpose” flocculants whose properties are tuned and optimised as required for different microalgal species and growth states, thereby allowing for greater control of harvesting processes. Once the optimal “fit-for-purpose” polymer flocculant (and dosing amount) is identified, the flocculant can then be applied in a desired harvesting environment.

Further aspects of the disclosure involve a convenient method and apparatus for harvesting microalgae. The optimal “fit-for-purpose” polymer flocculant determined according to the above aspect may be prepared, optionally in a continuous fashion, by mixing one or more monomers, a photoinitiator, water and a deoxygenation system, and exposing the mixture to UV light to initiate polymerisation. The optimal polymer may then be introduced to the suspension to be flocculated or harvested, in the pre-determined optimal dose.

The above method and apparatus for harvesting microalgae avoids the need for polymer batching, which is characterised by high chemical wastage due to overdose and polymer hydrolysis. The methods and apparatus of the disclosure provide polymeric flocculants in aqueous solution in ready-to-use form that can be prepared on-location. In addition, the apparatus used to carry out the methods may be assembled using cheap, readily available components, and is therefore highly cost-effective.

### Method for Harvesting Microalgae

In a first aspect of the disclosure there is provided a method for harvesting microalgae, the method comprising:
(i) mixing one or more monomers, a photoinitiator, water and a deoxygenation system to form a mixture;
(ii) exposing the mixture to UV light so as to form an aqueous solution of one or more polymers;
(iii) introducing the aqueous solution of one or more polymers to a suspension comprising microalgae in an amount sufficient to produce flocculated microalgae; and
(iv) harvesting the flocculated microalgae.

The method of the first aspect of the disclosure may take place in a flow reactor. A flow reactor is any reactor into which is fed a continuous flow of reactants, and from which emerges a continuous flow of product. This is in contrast to a batch reactor, into which is fed a single, predetermined amount of reactants to form a predetermined amount of product in a discrete step. Thus, the method of the first aspect of the disclosure may be a continuous method. A flow reactor is particularly advantageous for photochemical reactions due to increased photon transfer and mixing compared to a batch reactor. An example of a flow reactor suitable for the method of the first aspect of the disclosure is described below in relation to the third aspect of the disclosure.

Step (i) of the method of the first aspect of the disclosure involves mixing one or more monomers, a photoinitiator, water and a deoxygenation system to form a mixture.

The one or more monomers may be compounds comprising an alkene and a cationic moiety. The alkene is capable of reacting with the photoinitiator and taking part in a polymerisation reaction to form one or more polymers. The cationic moiety imparts a positive charge on the one or more polymers, enabling the flocculation of negatively charged microalgae. The cationic moiety may be a quaternary ammonium, phosphonium or sulfonium moiety. The quaternary ammonium, phosphonium or sulfonium moiety may be substituted by alkyl and/or aryl groups, particularly C₁ to C₂₀ alkyl groups and/or C₆ aryl groups. The one or more monomers may be selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC) and [3-(methacryloylamino)propyl]trimethylammonium chloride (MAmPTAC). In some embodiments the one or more monomers may be [2-(acryloyloxy)ethyl]trimethylammoniumchloride (AETAC). In some embodiments, the one or more monomers may be acrylamide and/or 2-(dimethylamino)ethyl acrylate (DMA).

In some cases, it may be desirable to adjust the charge density of the one or more polymers by incorporating co-monomers which do not contain a charged moiety. Thus, the method of the first aspect of the disclosure may further comprise mixing one or more co-monomers with the one or more monomers, the photoinitiator, the water and the deoxygenation system to form a mixture. The co-monomer may be a compound comprising an alkene, and not comprising a charged moiety. The alkene is capable of reacting with the photoinitiator and the one or more monomers, and taking part in a polymerisation reaction to form one or more co-polymers. The one or more co-monomers may be selected from the group consisting of methyl acrylate, methyl methacrylate, acrylamide, styrene, vinyl chloride, acrylonitrile and 2-(dimethylamino)ethyl acrylate (DMA).

The photoinitiator is a compound which produces a radical when exposed to UV light, and serves to initiate a polymerisation reaction in step (ii). Any suitable photoinitiator may be used. Examples of suitable photoinitiators include, but are not limited to, azobisisobutyronitrile (AIBN), benzoyl peroxide (BPO), 2,2-dimethoxy-2-phenylacetophenone (DMPA), camphorquinone (CQ), 2,2′-azobis(2-methylpropionamidine)dihydrochloride (V-50) and 4,4′-azobis(4-cyanovaleric acid) (V-501). In some embodiments the photoinitiator is DMPA.

Water is included in the mixture of step (i) of the method of the first aspect of the disclosure. The water acts as a solvent for the polymerisation reaction of step (iii), as well as solubilising the resulting one or more polymers.

The mixture of step (i) should be free from oxygen in order for the polymerisation reaction in step (ii) to occur, however, the components of the mixture may contain dissolved oxygen. Thus the mixture of step (i) includes a deoxygenation system. Deoxygenation systems are known in the art and comprise substances which react with, and deplete, any oxygen present in a mixture. The deoxygenation system may be an enzymatic deoxygenation system which comprises an enzyme and an enzyme substrate. In this case, the enzyme catalyses the reaction of the substrate and oxygen to form a product, resulting in the depletion of any oxygen present in the mixture. For example, in one commonly used deoxygenation system, the enzyme is glucose oxidase and the enzyme substrate is glucose.

In some embodiments, the mixture of step (i) does not contain a deoxygenation system but instead is subject to a deoxygenation process such as sparging with nitrogen or argon gas or the freeze-pump-thaw process. Thus, in another aspect of the disclosure there is provided a method for harvesting microalgae, the method comprising (i) mixing one or more monomers, a photoinitiator, and water to form a mixture; (ia) subjecting the mixture to a deoxygenation process; (ii) exposing the mixture to UV light so as to form an aqueous solution of one or more polymers; (iii) introducing the aqueous solution of one or more polymers to a suspension comprising microalgae in an amount sufficient to produce flocculated microalgae; and (iv) harvesting the flocculated microalgae. In this case, subjecting the mixture to a deoxygenation process may comprise adding a deoxygenation system to the mixture, sparging the mixture with nitrogen or argon gas, and/or subjecting the mixture to the freeze-pump-thaw process.

In step (ii) of the method of the first aspect of the disclosure, the mixture is exposed to UV light. UV light is understood to be light having a wavelength of between about 10 nm to 400 nm. The UV light may have a wavelength of between about 10 to 400 nm, or 50 to 400, 100 to 400, 200 to 400, or 300 to 400 nm. The UV light may have a wavelength of about 365 nm.

Exposing the mixture to UV light causes the photoinitiator to decompose to a radical species which initiates a polymerisation reaction of the one or more monomers (and optionally one or more co-monomers). The polymerisation occurs in water, such that the resulting polymer is dissolved in aqueous solution upon formation. Such an aqueous solution of the one or more polymers may be directly added to a suspension comprising microalgae, i.e. it is in ready-to-use form and avoids the disadvantages associated with batch processing.

In step (iii) of the method of the first aspect of the disclosure the aqueous solution of one or more polymers is introduced to a suspension comprising microalgae. In some embodiments, the method of the first aspect of the disclosure may take place in situ, that is, at the location of the suspension comprising microalgae to be harvested. The suspension comprising microalgae may be, for example, a body of water comprising microalgae. Examples of a body of water include a body of fresh water such as a dam, river, creek, lake, or pond; or a body of salt water such as a sea, ocean or estuary. The body of water may be, for example, a water tank or reservoir. That is, the aqueous solution of one or more polymers may be introduced directly to the suspension comprising microalgae. Thus, an advantage of the method of the first aspect of the disclosure is that it can be conveniently performed at any location. In some embodiments, the suspension comprising microalgae may be an aliquot comprising microalgae removed from a body of water comprising microalgae.

Microalgae are aquatic unicellular photosynthetic micro-organisms. In the context of this specification, microalgae includes eukaryotic organisms, as well as prokaryotic organisms such as cyanobacteria. Microalgae include freshwater and saltwater species. Examples of microalgae include Chaetoceros calcitrans, Chaetoceros gracilis, Nitzchia closterium, Phaeodactylum tricornutum, Thalassiosira pseudonana, Cylindrogheca fusiformis, Dunaliella tertiolecta, Tetraselmis suecica, Chlorella vulgaris, Dunaliella salina, Nannochloropsis oculate, Isochrysis galbana, Pavlova lutheri, Pavlova salina, Cryptomonas rufescens, Nostoc commune, Spirulina platensis, Aphanizomenon flos-aquae, Euglena gracilis, Rhodomonas salina, Porphyridium purpureum and Scendesmus sp. In some embodiments of the disclosure, the microalgae are selected from the group consisting of Chlorella vulgaris, Scendesmus sp, Rhodomonas salina, and Porphyridium purpureum.

The aqueous solution of the one or more polymers is introduced to the suspension comprising microalgae in an amount sufficient to produce flocculated microalgae. Producing flocculated microalgae may be understood to refer to achieving a flocculation efficiency of at least about 60%, or at least about 65, 70, 75, 80, 85, 90, 95, or 100%. Flocculation efficiency is as defined below. The skilled person will appreciate that the amount of polymer required to produce flocculated microalgae will vary depending on the nature of the polymer and the nature of the algae. For example, an amount of polymer sufficient to produce flocculated microalgae may be between about 10 to 500 mg/g of dry biomass or between about 10 to 50, 10 to 100, 10 to 200, 10 to 300, 10 to 400, 50 to 100, 50 to 200, 50 to 300, 50 to 400, 50 to 500, 100 to 200, 100 to 300, 100 to 400, 100 to 500, 200 to 300, 200 to 400, 200 to 500, 300 to 400, 300 to 500, or 400 to 500 mg/g of dry biomass. An amount of polymer sufficient to produce flocculated microalgae may be about 10 mg/g dry biomass, or about 50, 100, 200, 300, 400, or 500 mg/g dry biomass.

In step (iv) of the method of the disclosure, the flocculated microalgae is harvested. Harvesting the flocculated microalgae may involve separating the flocculated microalgae from the suspension comprising microalgae. Step (iv) may therefore comprise any suitable solid/liquid separation method such as, for example, one or more of filtration, centrifugation, sedimentation.

### Method for Preparing and Selecting a Polymeric Flocculant for Use in Harvesting Microalgae

In a second aspect of the disclosure there is provided a method for preparing and selecting a polymeric flocculant for use in harvesting microalgae, the method comprising:
(i) preparing a plurality of different polymers;
(ii) introducing an amount of each polymer into a separate suspension comprising microalgae;
(iii) repeating step (ii) at least once by varying the amount of each polymer so as to provide a series of separate suspensions comprising different polymers in different amounts;
(iv) measuring optical density of each suspension in the series;
(v) calculating a flocculation efficiency for each suspension in the series;
(vi) generating a flocculation efficiency dose-response curve for each polymer at each amount; and
(vii) selecting a polymeric flocculant.

In some embodiments, the method of the second aspect of the disclosure, and particularly steps (i) to (iii), may take place in situ, that is, at the location of the suspension comprising microalgae to be harvested. For example, if the suspension comprising microalgae to be harvested is located at a body of water such as a lake, steps (i) to (iii) may take place at the location of the body of water. Thus, an advantage of the method of the second aspect of the disclosure is that it can be conveniently performed at any location. In some embodiments, the method of the second aspect of the disclosure may take place at a separate location to the suspension comprising microalgae to be harvested, for example at a laboratory.

Step (i) of the second aspect of the disclosure involves preparing a plurality of different polymers. The plurality of different polymers may differ in molecular weight, composition, and/or charge density. For example, the plurality of different polymers may comprise two or more polymers having different molecular weights. In another example, the plurality of different polymers may comprise two or more polymers comprised of different monomers. In another example, the plurality of different polymers may comprise two or more polymers comprising the same cationic monomer, which is co-polymerised with different amounts of a co-monomer, resulting in different charge densities.

The plurality of different polymers may be prepared by a. mixing the one or more monomers, the photoinitiator, the water and the deoxygenation system to form a mixture; and b. exposing the mixture to UV light so as to form an aqueous solution of the polymer; wherein in step (ii) of the second aspect of the disclosure, introducing an amount of each polymer into a separate solution or suspension comprising microalgae comprises introducing an amount of the aqueous solution of the polymer. Steps a. and b. may take place in a flow reactor. A flow reactor is as described above in respect of the first aspect of the disclosure. As such, steps a. and b. may be performed in a continuous method.

In step a., the one or more monomers may be compounds comprising an alkene and a cationic moiety. The alkene is capable of reacting with the photoinitiator and taking part in a polymerisation reaction to form one or more polymers. The cationic moiety imparts a positive charge on the one or more polymers, enabling the flocculation of negatively charged microalgae. The cationic moiety may be a quaternary ammonium, phosphonium, or sulfonium moiety. The quaternary ammonium, phosphonium, or sulfonium moiety may be substituted by alkyl and/or aryl groups, particularly C₁ to C₂₀ alkyl groups and/or C₆ aryl groups. The one or more monomers may be selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC), and [3-(methacryloylamino)propyl]trimethylammonium chloride (MAmPTAC). In some embodiments the one or more monomers may be [2-(acryloyloxy)ethyl]trimethylammoniumchloride (AETAC). In some embodiments, the one or more monomers may be acrylamide and/or 2-(dimethylamino)ethyl acrylate (DMA).

It is possible to adjust the charge density of the one or more polymers by incorporating co-monomers which do not contain a charged moiety. Thus step a. may further comprise mixing one or more co-monomers with the one or more monomers, the photoinitiator, the water and the deoxygenation system to form a mixture. The co-monomer may be a compound comprising an alkene, and not comprising a charged moiety. The alkene is capable of reacting with the photoinitiator and the one or more monomers, and taking part in a polymerisation reaction to form one or more co-polymers. The one or more co-monomers may be selected from the group consisting of methyl acrylate, methyl methacrylate, acrylamide, styrene, vinyl chloride, acrylonitrile, and 2-(dimethylamino)ethyl acrylate (DMA).

The photoinitiator is a compound which produces a radical when exposed to UV light, and serves to initiate a polymerisation reaction in step b. Any suitable photoinitiator may be used in the method of the disclosure, and the selection of appropriate photoinitiators to suit a particular polymerisation reaction is well-known in the art. Examples of suitable photoinitiators include azobisisobutyronitrile (AIBN), benzoyl peroxide (BPO), 2,2-dimethoxy-2-phenylacetophenone (DMPA), and camphorquinone (CQ), 2,2′-azobis(2-methylpropionamidine)dihydrochloride (V-50), and 4,4′-azobis(4-cyanovaleric acid) (V-501). For example, a suitable photoinitiator may be DMPA.

Water is provided in step a. and acts as a solvent for the polymerisation reaction of step b., as well as solubilising the resulting one or more polymers.

The mixture of step a. should be free from oxygen in order for the polymerisation reaction in step b. to occur, however, the components of the mixture may contain dissolved oxygen. Thus the mixture of step a. includes a deoxygenation system. Deoxygenation systems are known in the art and comprise substances which react with and deplete any oxygen present in a mixture. The deoxygenation system may be an enzymatic deoxygenation system which comprises an enzyme and an enzyme substrate. In this case, the enzyme catalyses the reaction of the substrate and oxygen to form a product, resulting in the depletion of any oxygen present in the mixture. For example, in one commonly used deoxygenation system, the enzyme is glucose oxidase and the enzyme substrate is glucose.

In some embodiments, the mixture of step a. does not contain a deoxygenation system but instead is subject to a deoxygenation process such as sparging with nitrogen gas or the freeze-pump-thaw process. Thus, in one embodiment, the plurality of different polymers may be prepared by a. mixing the one or more monomers, the photoinitiator, and the water to form a mixture; ai. subjecting the mixture to a deoxygenation process; and b. exposing the mixture to UV light so as to form an aqueous solution of the polymer. In this case, subjecting the mixture to a deoxygenation process may comprise adding a deoxygenation system to the mixture, sparging the mixture with nitrogen or argon gas, and/or subjecting the mixture to the freeze-pump-thaw process.

In step b. of the method of the first aspect of the disclosure, the mixture is exposed to UV light. UV light is understood to be light having a wavelength of between about 10 nm to 400 nm. The UV light may have a wavelength of between about 10 to 400 nm, or 50 to 400, 100 to 400, 200 to 400, or 300 to 400 nm. The UV light may have a wavelength of about 365 nm.

Exposing the mixture to UV light causes the photoinitiator to decompose to a radical species which initiates a polymerisation reaction of the one or more monomers (and optionally one or more co-monomers). The polymerisation occurs in water, such that the resulting polymer is dissolved in aqueous solution upon formation. Such an aqueous solution of the one or more polymers may be directly added to a suspension comprising microalgae, i.e. it is in ready to use form and avoids the disadvantages associated with batch processing.

When the plurality of different polymers is prepared by a. mixing the one or more monomers, the photoinitiator, the water and the deoxygenation system to form a mixture; and b. exposing the mixture to UV light so as to form an aqueous solution of the polymer, preparing a plurality of different polymers differing in molecular weight may be achieved by exposing the mixture to UV light for varying amounts of time and/or changing the concentration of one or more monomers in the mixture. Preparing a plurality of different polymers differing in composition may be achieved by selecting different monomers. Preparing a plurality of different polymers differing in charge density may be achieved by including one or more co-monomers (optionally in varying amounts) in the mixture.

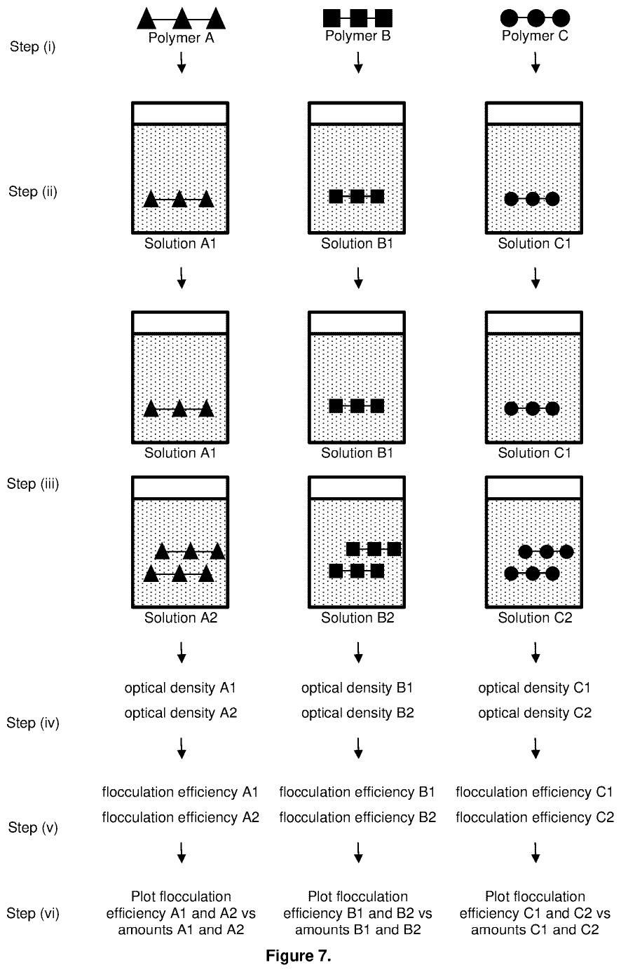

In step (ii) of the method of the second aspect of the disclosure each of the plurality of different polymers is introduced into a separate suspension of microalgae in an amount. See step (ii) of FIG. 7. That is, at least two separate suspensions are prepared, each containing a different polymer in a first amount (for example, a suspension comprising polymer A at 10 mg/g dry biomass and a suspension comprising polymer B at 10 mg/g dry biomass). See, for example, solutions A1 and B1 of FIG. 7.

In step (iii) of the method of the second aspect of the disclosure, step (ii) is repeated at least once, or may be repeated at least 2, 3, 4, 5, 6, 7, 8, 9, or 10 times. Each time step (ii) is repeated, separate suspensions comprising each of the plurality of different polymers, in a different amount, are prepared. That is, where step (iii) involves repeating step (ii) once, the series of suspensions comprises the at least two separate suspensions, each containing a different polymer in a first amount (for example, a suspension comprising polymer A at 10 mg/g dry biomass and a suspension comprising polymer B at 10 mg/g dry biomass), and a further at least two suspensions, each containing a different polymer in a second amount (for example, a suspension comprising polymer A at 50 mg/g dry biomass and a suspension comprising polymer B at 50 mg/g dry biomass). See, for example, FIG. 7 where in step (iii), step (ii) has been repeated once to form a series of solutions of Polymer A (Solution A1 containing Polymer A in a first amount, and Solution A2, containing Polymer A in a second amount) and a series of solutions of Polymer B (Solution B1 containing Polymer B in a first amount, and Solution B2, containing Polymer B in a second amount). Increasing the number of repetitions allows for the preparation of a more detailed dose-response curve.

In step (iv) of the method of the second aspect of the disclosure, the optical density of each suspension in the series is measured. See FIG. 7 where in step (iv) an optical density is determined for each of solutions A1, A2, B1, B2, etc.). Optical density is defined to be the logarithm of the ratio of the intensity of light falling upon a suspension and the intensity transmitted through the suspension. Optical density may be measured using a UV spectrophotometer. A suitable wavelength for measuring optical density is 680 nm.

In step (v) of the method of the second aspect of the disclosure, the flocculation efficiency for each suspension in the series is calculated. See FIG. 7 where in step (iv) a flocculation efficiency is determined for each of solutions A1, A2, B1, B2, etc.). Flocculation efficiency may be calculated using the following equation:

$$Flocculation efficiency ( \% ) = ( \frac{{OD}_{i} - {OD}_{f}}{{OD}_{i}} ) \times 100$$

wherein ODᵢ is the optical density of the suspension prior to introduction of the polymer in step (ii), and OD_{f} is the optical density of the solution or suspension after introduction of the polymer in step (ii).

In step (vi) of the method of the second aspect of the disclosure, a flocculation efficiency dose-response curve for each polymer at each amount is generated. That is, for each of the plurality of different polymers, the flocculation efficiency is plotted on the y-axis of a graph, against the amount of the polymer added in step (ii) on the x-axis. See FIG. 7 where in step (vi) the flocculation efficiency for solutions A1 and A2 is plotted against amounts A1 and A2, generating a dose-response curve for the series of solutions of Polymer A (similarly for solutions of Polymer B, etc.).

In step (vii) of the method of the second aspect of the disclosure, the most suitable polymeric flocculant for use in harvesting microalgae is selected. The polymeric flocculant may be selected based on the dose-response curves prepared in step (vi). For example, the dose-response curves may reveal which of the plurality of different polymers has the highest flocculation efficiency, and what amount of the polymer is required to be added to a suspension comprising microalgae to achieve that flocculation efficiency. Other factors may also be taken into account when selecting the polymeric flocculant, such as the amount of polymer residue not aggregated with the microalgae present in the suspension, the reusability of the water in the suspension following microalgae harvesting for repeated microalgae cultivation, and the rate of polymer hydrolysis after harvesting.

In another aspect of the disclosure, there is provided a method for harvesting microalgae using an optimised polymeric flocculant, the method comprising:
a. identifying the optimised polymeric flocculant, by:
   (a.i.) preparing a plurality of different polymers;
   (a.ii.) introducing an amount of each polymer into a separate suspension comprising microalgae;
      (a.iii.) repeating step (a.ii.) at least once by varying the amount of each polymer so as to provide a series of separate suspensions comprising different polymers in different amounts;
      (a.iv.) measuring optical density of each suspension in the series;
      (a.v.) calculating a flocculation efficiency for each suspension in the series;
      (a.vi.) generating a flocculation efficiency dose-response curve for each polymer at each amount; and
      (a.vii.) selecting an optimised polymeric flocculant;
then
b. harvesting microalgae using the optimised polymeric flocculant, by:
   (b.i.) preparing the optimised polymeric flocculant, by:
      (b.i.i.) mixing one or more monomers, a photoinitiator, water and a deoxygenation system to form a mixture;
      (b.i.ii.) exposing the mixture to UV light so as to form an aqueous solution of the optimised polymeric flocculant;
   (b.ii.) introducing the aqueous solution of the optimised polymeric flocculant to a suspension comprising microalgae in an amount sufficient to produce flocculated microalgae; and
   (b.iii.) harvesting the flocculated microalgae.

In the above aspect of the disclosure, the features of steps a. are as described above in relation to the second aspect of the disclosure and the features of steps b. are as described above in relation to the first aspect of the disclosure.

### Apparatus for Flocculating a Suspension of Particles

In a third aspect of the disclosure there is provided an apparatus for flocculating an aqueous suspension of particles, the apparatus comprising:
a plurality of vessels for containing one or more monomers, a photoinitiator, water and a deoxygenation system;
a mixing vessel for mixing the one or more monomers, the photoinitiator, the water and the deoxygenation system;
a polymerisation vessel for producing an aqueous solution of a polymer; and
a UV light source for irradiating the polymerisation vessel;
wherein,
   the plurality of vessels are in fluid communication with the mixing vessel, and the mixing vessel is in fluid communication with the polymerisation vessel.

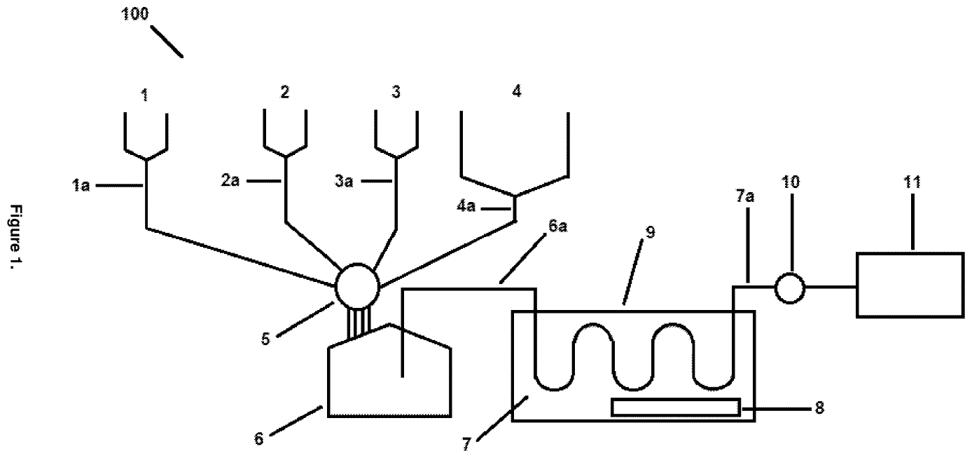

FIG. 1 depicts an apparatus 100 in accordance with one embodiment of the disclosure. Apparatus 100 comprises a plurality of vessels 1, 2, 3 and 4 for containing the one or more monomers, the photoinitiator, the water and the deoxygenation system, which are in fluid communication with mixing vessel 6 via conduits 1a, 2a, 3a and 4a. A first pump 5 is located between the plurality of vessels 1, 2, 3 and 4 and mixing vessel 6. In some embodiments, the first pump 5 is a positive displacement pump, such as a peristaltic pump.

Mixing vessel 6 is in fluid communication with polymerisation vessel 7 via conduit 6a. Polymerisation vessel 7 may be in the form of a tube. Optionally the tube may be permeable to UV radiation. Polymerisation vessel 7 and UV light source 8 are contained within container 9. Container 9 serves to isolate the polymerisation vessel 7 and the UV light source 8 from visible light.

Polymerisation vessel 7 is in fluid communication with flocculation vessel 11 via conduit 7a. A second pump 10 is located between the polymerisation vessel 7 and flocculation vessel 11. In some embodiments, pump 10 is a dosing pump. Alternatively, the apparatus may not contain a flocculation vessel, but instead the conduit 7a and second pump 10 may transfer the aqueous solution of a polymer directly into a body of water.

In use, the plurality of vessels 1, 2, 3 and 4 are charged with the one or more monomers, the photoinitiator, the water and the deoxygenation system. Pump 5 is then activated to pump desired amounts of each component into the mixing vessel 6 via conduits 1a, 2a, 3a and 4a. The mixture produced in mixing vessel 6 is then transported to polymerisation vessel 7 via conduit 6a. UV light source 8 is activated resulting in polymerisation of the one or more monomers in polymerisation vessel 7. Pump 10 is then activated to pump the polymers produced in polymerisation vessel 7 (as a solution in water) to flocculation vessel 11 via conduit 7a.

In the apparatus of the third aspect of the disclosure, the particles may be selected from the group consisting of mud, silt, clay, microalgae, mine tailings, and particles found in water. The particles may be microalgae.

### Uses of the Apparatus for Flocculating Particles

In a fourth aspect of the disclosure there is provided a use of the apparatus of the third aspect of the disclosure for flocculating suspended particles. The particles may be selected from the group consisting of mud, silt, clay, microalgae, and mine tailings. The particles may be microalgae.

In a fifth aspect of the disclosure there is provided the apparatus of the third aspect of the disclosure when used for flocculating suspended particles. The particles may be selected from the group consisting of mud, silt, clay, microalgae, and mine tailings. The particles may be microalgae.

The present disclosure may be described by reference to the following numbered embodiments.
1. A method for harvesting microalgae, the method comprising:
   (i) mixing one or more monomers, a photoinitiator, water and a deoxygenation system to form a mixture;
   (ii) exposing the mixture to UV light so as to form an aqueous solution of one or more polymers;
   (iii) introducing the aqueous solution of one or more polymers to a suspension comprising microalgae in an amount sufficient to produce flocculated microalgae; and
   (iv) harvesting the flocculated microalgae.
2. The method of embodiment 1, wherein steps (i) and (ii) take place in a flow reactor.
3. The method of embodiment 1 or embodiment 2, wherein the method is a continuous method.
4. The method of any one of embodiments 1 to 3, wherein the one or more monomers are compounds comprising an alkene and a cationic moiety.
5. The method of any one of embodiments 1 to 4, wherein the one or more monomers are selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC), and [3-(methacryloylamino)propyl]trimethylammonium chloride (MAmPTAC).
6. The method of any one of embodiments 1 to 5, wherein step (i) further comprises mixing one or more co-monomers with the one or more monomers, the photoinitiator, the water and the deoxygenation system to form a mixture.
7. The method of embodiment 6, wherein the one or more co-monomers are selected from the group consisting of methyl acrylate, methyl methacrylate, acrylamide, styrene, vinyl chloride, acrylonitrile, and 2-(dimethylamino)ethyl acrylate (DMA).
8. The method of any one of embodiments 1 to 7, wherein the photoinitiator is selected from the group consisting of azobisisobutyronitrile (AIBN), benzoyl peroxide (BPO), 2,2-dimethoxy-2-phenylacetophenone (DMPA), camphorquinone (CQ), 2,2′-azobis(2-methylpropionamidine)dihydrochloride (V-50), and 4,4′-azobis(4-cyanovaleric acid) (V-501).
9. The method of any one of embodiments 1 to 8, wherein the deoxygenation system is an enzymatic deoxygenation system.
10. The method of embodiment 9, wherein the enzymatic deoxygenation system comprises an enzyme and an enzyme substrate.
11. The method of embodiment 10, wherein the enzyme is glucose oxidase and the enzyme substrate is glucose.
12. The method of any one of embodiments 1 to 11, wherein harvesting the flocculated microalgae comprises filtering the suspension comprising microalgae to separate the flocculated microalgae.
13. The method of any one of embodiments 1 to 12, wherein the method takes place in situ.
14. A method for preparing and selecting a polymeric flocculant for use in harvesting microalgae, the method comprising:
   (i) preparing a plurality of different polymers;
   (ii) introducing an amount of each polymer into a separate suspension comprising microalgae;
   (iii) repeating step (ii) at least once by varying the amount of each polymer so as to provide a series of separate suspensions comprising different polymers in different amounts;
   (iv) measuring optical density of each suspension in the series;
   (v) calculating a flocculation efficiency for each suspension in the series;
   (vi) generating a flocculation efficiency dose-response curve for each polymer at each amount; and
   (vii) selecting a polymeric flocculant.
15. The method of embodiment 14, wherein in the step (vii) the polymeric flocculant is selected based on the dose response curves.
16. The method of embodiment 14 or embodiment 15, wherein steps (i) to (iii) take place in situ.
17. The method of embodiment 14 or embodiment 16, wherein step (ii) may be repeated at least 2, 3, 4, 5, 6, 7, 8, 9, or 10 times.
18. The method of any one of embodiments 14 to 17, wherein the different polymers may differ in molecular weight, composition, and/or charge density.
19. The method of any one of embodiments 14 to 18, wherein in step (i), each of the plurality of different polymers is prepared by:
   a. mixing the one or more monomers, the photoinitiator, the water and the deoxygenation system to form a mixture; and
   b. exposing the mixture to UV light so as to form an aqueous solution of the polymer;
wherein in step (ii), introducing an amount of each polymer into a separate suspension comprising microalgae comprises introducing an amount of the aqueous solution of the polymer.
20. The method of embodiment 19, wherein steps a. and b. take place in a flow reactor.
21. The method of embodiment 19 or embodiment 20, wherein the one or more monomers are compounds comprising an alkene and a cationic moiety.
22. The method of any one of embodiments 19 to 21, wherein the one or more monomers are selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC), and [3-(methacryloylamino)propyl]trimethylammonium chloride (MAmPTAC).
23. The method of any one of embodiments 19 to 22, wherein step a. further comprises mixing one or more co-monomers with the one or more monomers, the photoinitiator, the water and the deoxygenation system to form a mixture.
24. The method of embodiment 23, wherein the one or more co-monomers are selected from the group consisting of methyl acrylate, methyl methacrylate, acrylamide, styrene, vinyl chloride, acrylonitrile, and 2-(dimethylamino)ethyl acrylate (DMA).
25. The method of any one of embodiments 19 to 24, wherein the photoinitiator is selected from the group consisting of azobisisobutyronitrile (AIBN), benzoyl peroxide (BPO), 2,2-dimethoxy-2-phenylacetophenone (DMPA), camphorquinone (CQ), 2,2′-azobis(2-methylpropionamidine)dihydrochloride (V-50), and 4,4′-azobis(4-cyanovaleric acid) (V-501).
26. The method of any one of embodiments 19 to 25, wherein the deoxygenation system is an enzymatic deoxygenation system.
27. The method of embodiment 26, wherein the enzymatic deoxygenation system comprises an enzyme and an enzyme substrate.
28. The method of embodiment 27, wherein the enzyme is glucose oxidase and the enzyme substrate is glucose.
29. The method of any one of embodiments 14 to 28, wherein step (v) may comprise calculating the flocculation efficiency according to the following equation:

$$Flocculation efficiency ( \% ) = ( \frac{{OD}_{i} - {OD}_{f}}{{OD}_{i}} ) \times 100$$

wherein ODᵢ is the optical density of the suspension prior to introduction of the polymer in step (ii), and OD_{f} is the optical density of the suspension after introduction of the polymer in step (ii).
30. An apparatus for flocculating an aqueous suspension of particles using one or more polymers, the apparatus comprising:
   a plurality of vessels for containing one or more monomers, a photoinitiator, water and a deoxygenation system;
   a mixing vessel for mixing the one or more monomers, the photoinitiator, the water and the deoxygenation system to form a mixture;
   a polymerisation vessel for producing an aqueous solution of a polymer; and
   a UV light source for irradiating the polymerisation vessel;
wherein the plurality of vessels are in fluid communication with the mixing vessel, and the mixing vessel is in fluid communication with the polymerisation vessel.
31. The apparatus of embodiment 30, further comprising a first pump for transferring the one or more monomers, the photoinitiator, the water and the deoxygenation system from the plurality of vessels to the mixing vessel.
32. The apparatus of embodiment 30, wherein the first pump for transferring the one or more monomers, the photoinitiator, the water and the deoxygenation system from the plurality of vessels to the mixing vessel is a positive displacement pump.
33. The apparatus of embodiment 30 or embodiment 31, wherein the first pump for transferring the one or more monomers, the photoinitiator, the water and the deoxygenation system from the plurality of vessels to the mixing vessel is a peristaltic pump.
34. The apparatus of any one of embodiments 30 to 33, further comprising a second pump for transferring the aqueous solution of the polymer from the polymerisation vessel.
35. The apparatus of embodiment 34, wherein the second pump is for transferring the aqueous solution of the polymer from the polymerisation vessel to a body of water.
36. The apparatus of embodiment 34, further comprising a flocculation vessel for containing a suspension of particles, wherein the polymerisation vessel is in fluid communication with the flocculation vessel, and wherein the second pump is for transferring the aqueous solution of the polymer from the polymerisation vessel to the flocculation vessel.
37. The apparatus of embodiment 34 or embodiment 35 wherein the second pump is a dosing pump.
38. The apparatus of any one of embodiments 30 to 37, wherein the polymerisation vessel is in the form of a tube which is permeable to UV radiation.
39. The apparatus of any one of embodiments 30 to 38, wherein the polymerisation vessel and the UV light source are located within a container which excludes visible light.
40. The apparatus of any one of embodiments 30 to 39, wherein the suspended particles are microalgae.
41. Use of the apparatus of any one of embodiments 30 to 40 for flocculating suspended particles.
42. The apparatus of any one of embodiments 30 to 40 when used for flocculating suspended particles.
43. The use of embodiment 41 or the apparatus of embodiment 42, wherein the suspended particles are selected from the group consisting of mud, silt, clay, microalgae, mine tailings, and particles found in wastewater.
44. The use of embodiment 41, the apparatus of embodiment 42, or the use or apparatus of claim 43 wherein the suspended particles are microalgae.

### Examples

The present disclosure is further described below by reference to the following non-limiting examples.

Microalgae were harvested using the general method described below.

### Method

10 mL of an aqueous solution containing 2,2-dimethoxy-2-phenylacetophenone (DMPA) (5 mg), glucose oxidase (1 mg), D-glucose (100 mg) and various amounts of the monomer 2-(acryloyloxy)ethyl]trimethylammonium chloride (AETAC) were transferred to the reaction tube and the mixture remained steady for 30 min. A UV-lamp (9 W×4 with λₘₐₓ=365 nm and 3.5 mW/cm²) was then turned on in order to commence polymerisation. After 30 minutes of irradiation the polymerisation was terminated by switching off the UV-lamp.

The monomer concentrations were 120 mg/g, 240 mg/g and 360 mg/g, and the resulting polymers were designated as P-120, P-240 and P-360, respectively. The polymers were obtained as gels and then dissolved in Milli-Q water and stirred for 60 minutes to 0.24% wt/v. The solutions were stored at room temperature and used within 1 day of preparation.

Table 1 sets out the properties of the synthesised polymers and their stock solutions.

TABLE 1

| | Monomer | Stock | Zeta |
|---|---|---|---|
| | conversion | solution | potential |
| | (%) | (g/L) | (mV) |
| P-120 | 99 | 2.4 | 69.8 |
| P-240 | 96 | 2.4 | 55.8 |
| P-360 | 83 | 2.4 | 67.6 |

Flocculation experiments were conducted using a multi-flask orbital shaker. The microalgal suspension (100 mL) was added to a 250 mL flask. A specified polymer solution volume was then introduced to the microalgal suspension mixed at 200 rpm. Then, the mixing rate was reduced to 50 rpm for 5 min, followed by 1 h settling. Optical density was measured before and after the experiment to determine the flocculation efficiency.

The optimal dose of the polymer was determined by a dose-response relationship experiment. Polymer doses (4-36 mg/L) were added to the microalgal suspension for flocculation experiments. The polymer doses were subsequently normalised against dry biomass concentration.

After flocculation, an aliquot from half the height of the 100 mL flask was taken to evaluate the flocculation effect using a UV spectrophotometer at OD 680 nm. The flocculation efficiency was calculated based on the change in the optical density at a wavelength of 680 nm before and after each polymer addition, as shown in the following equation:

$$Flocculation efficiency ( \% ) = ( \frac{{OD}_{i} - {OD}_{f}}{{OD}_{i}} ) \times 100$$

wherein ODᵢ and OD_{f} are the optical densities of the culture before and after polymer addition.

All experiments were conducted with two technical replicates using one biological replicate of the microalgae culture. The dry biomass concentration was measured by filtering a 100 mL aliquot microalgal culture through a 0.45 μm pre-weighed glass fibre filter paper.

The charges of all polymers and microalgae cultures were measured using a Zetasizer nano instrument (Nano ZS Zen 3600, Malvern, UK).

### Results

Table 2 sets out the properties of the microalgal cultures that were harvested.

TABLE 2

| | | |
|---|---|---|
| Optical density | Dry weight | Zeta potential |
| (at 600 nm) | (g/L) | (mV) |

Freshwater species

| | | | |
|---|---|---|---|
| Chlorella vulgaris | 1.26 | 0.16 | −13.7 |
| Scenedesmus sp | 0.33 | 0.09 | −23.2 |

Marine species

| | | | |
|---|---|---|---|
| Rhodomonas salina | 0.54 | 0.34 | −7.93 |
| Porphyridium | 0.27 | 0.31 | −12.1 |
| purpureum | | | |

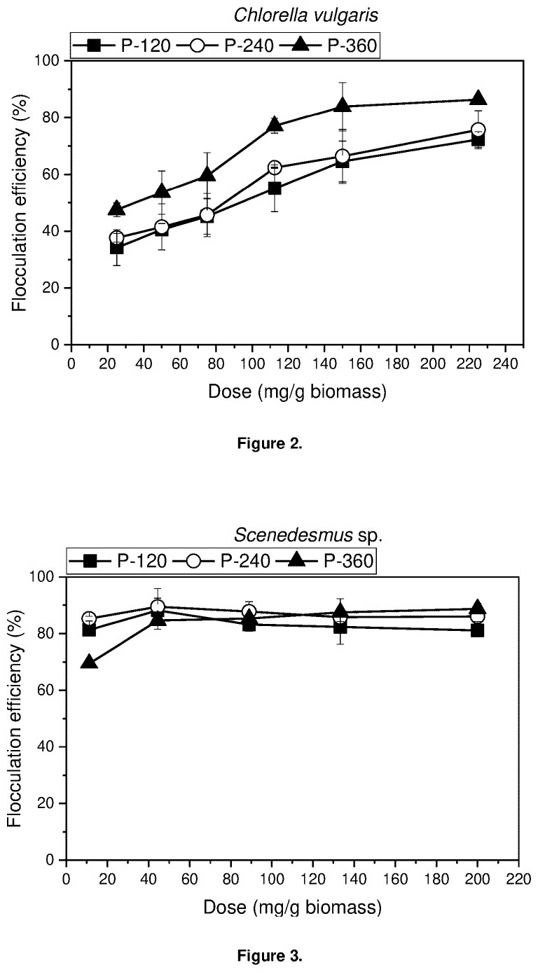

FIG. 2 shows the flocculation efficiency of the three polymers with Chlorella vulgaris. It was found that polymer P-360 worked best for Chlorella vulgaris, with >80% efficiency and >100 mg/g of dry biomass. Lower molecular weight polymers P-120 and P-240 were not as effective at flocculating Chlorella vulgaris. Their maximum efficiencies were ca. 70% at a dose of 225 mg polymer/g dry biomass. All three polymers started reaching plateau efficiency at 150 mg/g of dry biomass.

FIG. 3 shows the flocculation efficiency of the three polymers with Scenedesmus sp. P-120 worked best for Scenedesmus sp., with the optimal dose being ˜50 mg/g dry biomass. All three polymers showed similar performance for Scenedesmus sp. at high doses (>50 mg/g dry biomass), but no significant increase in efficiency. Without adding polymer, ˜50% of Scenedesmus sp. is settled after 1 hour. Adding polymer and after 1 hr settling, the flocculation efficiency is increased by 35-40% to >80%. After flocculation at optimal dose, the supernatant from both cultures became positively charged.

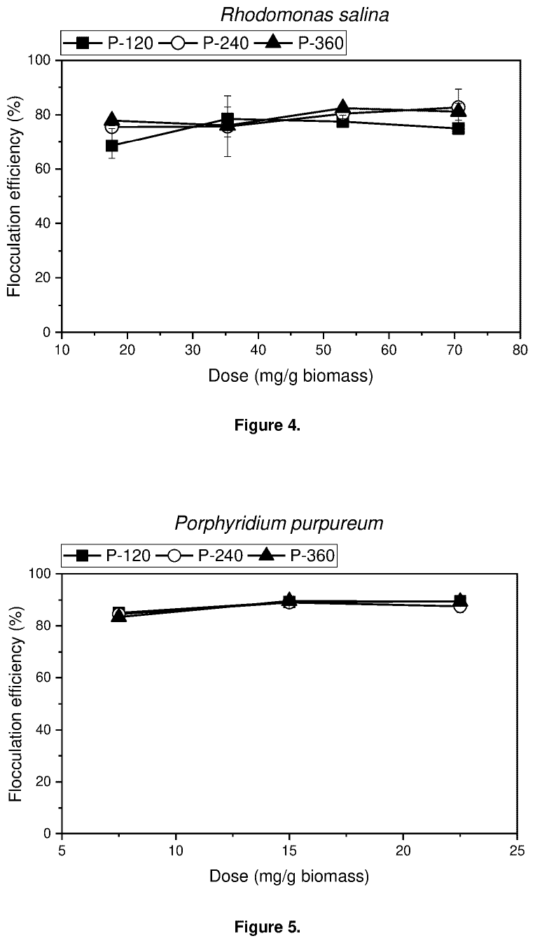

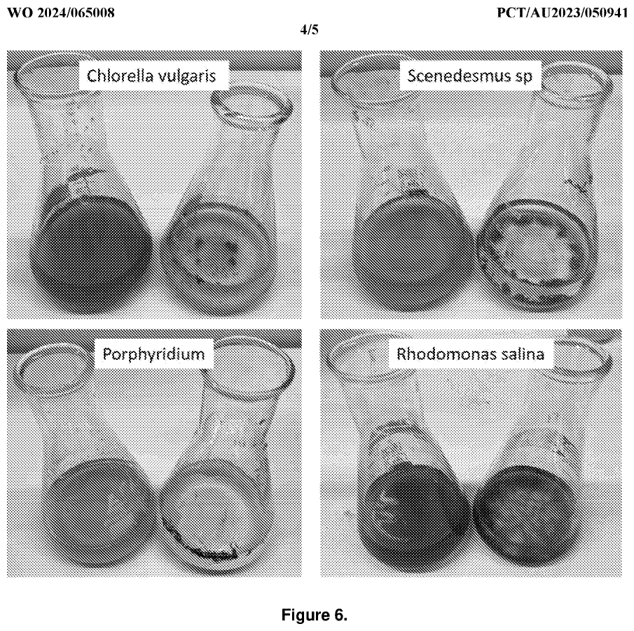

FIG. 4 shows the flocculation efficiency of the three polymers with Rhodomonas salina. All three polymers are similarly effective at flocculating Rhodomonas salina.

Flocculation efficiencies above 80% were achieved at 35 mg/g dry biomass, with further increases in polymer dose not resulting in any further increases in efficiency.

FIG. 5 shows the flocculation efficiency of the three polymers with Porphyridium purpureum. All three polymers are similarly effective at flocculating Porphyridium purpureum. Flocculation efficiencies of 83-90% were achieved at 7.5-22.5 mg/g dry biomass, with further increases in polymer dose not resulting in any further increases in efficiency. Flocculation occurred immediately without requiring any significant settling time.

Table 3 below shows the Zeta potential (mV) of microalgal culture after flocculation with the three polymers.

TABLE 3

| | | Scenedesmus | Rhodomonas | Porphyridium |
|---|---|---|---|---|
| | C. vulgaris | sp | salina | purpureum |
| Initial | −13.7 | −23.2 | −7.93 | −12.1 |
| P-120 | 9.6 | 17.3 | −3.01 | −9.25 |
| P-240 | 16.6 | 17.7 | −1.95 | −9.87 |
| P-360 | 17.8 | 16.7 | −1.14 | −11.1 |

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of an two or more of said steps, features, compositions and compounds.

1. A method for harvesting microalgae, the method comprising:
(i) mixing one or more monomers, a photoinitiator, water and a deoxygenation system to form a mixture;
(ii) exposing the mixture to UV light so as to form an aqueous solution of one or more polymers;
(iii) introducing the aqueous solution of one or more polymers to a suspension comprising microalgae in an amount sufficient to produce flocculated microalgae; and
(iv) harvesting the flocculated microalgae.

2. The method of claim 1, wherein steps (i) and (ii) take place in a flow reactor.

3. The method of claim 1 or claim 2, wherein the one or more monomers are compounds comprising an alkene and a cationic moiety.

4. The method of any one of claims 1 to 3, wherein the one or more monomers are selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC), and [3-(methacryloylamino)propyl]trimethylammonium chloride (MAmPTAC).

5. The method of any one of claims 1 to 4, wherein step (i) further comprises mixing one or more co-monomers with the one or more monomers, the photoinitiator, the water and the deoxygenation system to form a mixture.

6. The method of claim 5, wherein the one or more co-monomers are selected from the group consisting of methyl acrylate, methyl methacrylate, acrylamide, styrene, vinyl chloride, acrylonitrile, and 2-(dimethylamino)ethyl acrylate (DMA).

7. The method of any one of claims 1 to 6, wherein the deoxygenation system is an enzymatic deoxygenation system, the enzymatic deoxygenation system comprising an enzyme and an enzyme substrate.

8. The method of any one of claims 1 to 7, wherein the method takes place in situ.

9. A method for preparing and selecting a polymeric flocculant for use in harvesting microalgae, the method comprising:
(i) preparing a plurality of different polymers;
(ii) introducing an amount of each polymer into a separate suspension comprising microalgae;
(iii) repeating step (ii) at least once by varying the amount of each polymer so as to provide a series of separate suspensions comprising different polymers in different amounts;
(iv) measuring optical density of each suspension in the series;
(v) calculating a flocculation efficiency for each suspension in the series;
(vi) generating a flocculation efficiency dose-response curve for each polymer at each amount; and
(vii) selecting a polymeric flocculant.

10. The method of claim 9, wherein steps (i) to (iii) take place in situ.

11. The method of claim 9 or claim 10, wherein step (ii) may be repeated at least 2, 3, 4, 5, 6, 7, 8, 9, or 10 times.

12. The method of any one of claims 9 to 11, wherein in step (i), each of the plurality of different polymers is prepared by:
a. mixing the one or more monomers, the photoinitiator, the water and the deoxygenation system to form a mixture; and
b. exposing the mixture to UV light so as to form an aqueous solution of the polymer;
wherein in step (ii), introducing an amount of each polymer into a separate suspension comprising microalgae comprises introducing an amount of the aqueous solution of the polymer.

13. The method of claim 12, wherein steps a. and b. take place in a flow reactor.

14. The method of claim 12 or claim 13, wherein the one or more monomers are compounds comprising an alkene and a cationic moiety.

15. The method of any one of claims 12 to 14, wherein the one or more monomers are selected from the group consisting of [2-(acryloyloxy)ethyl]trimethylammoniumchloride (AETAC), (3-acrylamidopropyl)trimethylammonium chloride (AmPTAC), diallyldimethylammonium chloride (DADMAC), [2-(methacryloyloxy)ethyl]trimethylammonium chloride (MAETAC), and [3-(methacryloylamino)propyl]trimethylammonium chloride (MAmPTAC).

16. The method of any one of claims 12 to 15, wherein step a. further comprises mixing one or more co-monomers with the one or more monomers, the photoinitiator, the water and the deoxygenation system to form a mixture.

17. The method of claim 16, wherein the one or more co-monomers are selected from the group consisting of methyl acrylate, methyl methacrylate, acrylamide, styrene, vinyl chloride, acrylonitrile, and 2-(dimethylamino)ethyl acrylate (DMA).

18. The method of any one of claims 12 to 17, wherein the deoxygenation system is an enzymatic deoxygenation system, the enzymatic deoxygenation system comprising an enzyme and an enzyme substrate.

19. The method of any one of claims 9 to 18, wherein step (v) may comprise calculating the flocculation efficiency according to the following equation:
$$Flocculation efficiency ( \% ) = ( \frac{{OD}_{i} - {OD}_{f}}{{OD}_{i}} ) \times 100$$
wherein ODᵢ is the optical density of the suspension prior to introduction of the polymer in step (ii), and OD_{f} is the optical density of the suspension after introduction of the polymer in step (ii).

20. An apparatus for flocculating an aqueous suspension of particles using one or more polymers, the apparatus comprising:
a plurality of vessels for containing one or more monomers, a photoinitiator, water and a deoxygenation system;
a mixing vessel for mixing the one or more monomers, the photoinitiator, the water and the deoxygenation system to form a mixture;
a polymerisation vessel for producing an aqueous solution of a polymer; and
a UV light source for irradiating the polymerisation vessel;
wherein the plurality of vessels are in fluid communication with the mixing vessel, and the mixing vessel is in fluid communication with the polymerisation vessel.

21. The apparatus of claim 20, further comprising a first pump for transferring the one or more monomers, the photoinitiator, the water and the deoxygenation system from the plurality of vessels to the mixing vessel.

22. The apparatus of claim 20 or claim 21, further comprising a second pump for transferring the aqueous solution of the polymer from the polymerisation vessel.

23. The apparatus of claim 22, further comprising a flocculation vessel for containing a suspension of particles, wherein the polymerisation vessel is in fluid communication with the flocculation vessel, and wherein the second pump is for transferring the aqueous solution of the polymer from the polymerisation vessel to the flocculation vessel.

24. The apparatus of any one of claims 20 to 23, wherein the polymerisation vessel is in the form of a tube which is permeable to UV radiation.

25. The apparatus of any one of claims 20 to 24, wherein the suspended particles are microalgae.
